SEQUENCE LISTING (8 sequences)

SEQ ID NO: 1 - DNA, 250 bp, synthetic construct
  source 1..250: mol_type = other DNA
ttgccgacgatcgggcagagggaacggcggccaggtctaccgcgtctgcgacgggactttggggcccgccgtggggctggaggtagactagcagctcgtgccacggctcggaaggtgtggtaggcgtcgctgtttctctgtgcctccagttaggtcgaggtaaaagtcggttgcgctcgggtgcctcgccgcggtagcctggaaaaggacccgcgtcgtttctgaggcaaggacttttctgtccgtctat

SEQ ID NO: 2 - DNA, 500 bp, synthetic construct
  source 1..500: mol_type = other DNA
gggagcgcgcgcgcgcgcgcgtttgcgaaggcgcgcgcccgtcggtgggtcgagcggttcccgagcggttccgatggcgcggaaagcgtcggaaattcccggaaaggtccgaaaaagcccgaaaaggtccgaaaagtgccggaagggcccgctcgaaaagcgaaggaaggcgggccggcggtgttggttcccggccggcgggtctccccgtgtcctcctgaggttgccaggtctacccgcgcgaaggagtttgccgacgatcgggcagagggaacggcggccaggtctaccgcgtctgcgacgggactttggggcccgccgtggggctggaggtagactagcagctcgtgccacggctcggaaggtgtggtaggcgtcgctgtttctctgtgcctccagttaggtcgaggtaaaagtcggttgcgctcgggtgcctcgccgcggtagcctggaaaaggacccgcgtcgtttctgaggcaaggacttttctgtccgtctat

SEQ ID NO: 3 - DNA, 750 bp, synthetic construct
  source 1..750: mol_type = other DNA
gaaggcgggccggcggtgttggttcccggccggcgggtctccccgtgtcctcctgaggttgccaggtctaccgtataccccgtagcctcgtctgccggtccttccgcggccgcctggtctccccgtagcattctgcggccgccaggtctacccggccgcggtcgccatgtttgttttgccggttggcgacctgtaggggtcgctgcaggtccggccgcgagaacggcgttggggtctgcccggcagcgacgggagcgcgcgcgcgcgcgcgtttgcgaaggcgcgcgcccgtcggtgggtcgagcggttcccgagcggttccgatggcgcggaaagcgtcggaaattcccggaaaggtccgaaaaagcccgaaaaggtccgaaaagtgccggaagggcccgctcgaaaagcgaaggaaggcgggccggcggtgttggttcccggccggcgggtctccccgtgtcctcctgaggttgccaggtctacccgcgcgaaggagtttgccgacgatcgggcagagggaacggcggccaggtctaccgcgtctgcgacgggactttggggcccgccgtggggctggaggtagactagcagctcgtgccacggctcggaaggtgtggtaggcgtcgctgtttctctgtgcctccagttaggtcgaggtaaaagtcggttgcgctcgggtgcctcgccgcggtagcctggaaaaggacccgcgtcgtttctgaggcaaggacttttctgtccgtctat

SEQ ID NO: 4 - DNA, 1000 bp, synthetic construct
  source 1..1000: mol_type = other DNA
tcgactcagcagcaaaatcagcgagacagtctccaaaggaagcctagcacagcacaggagagcagagcaatgcggatgaaagctgagcaagacaaagcagagccctgctagggaaacgacggcggggttggttcccggccggcaggtgttccccggctcgggcgcggaaagcgtcggaaattcccggaaaggtccgaaaggacccgaaaaagttcgaaaagtgtcggaagggcccgttcgaaaagcagaggaaggcgggccggcggtgttggttcccggccggcgggtctccccgtgtcctcctgaggttgccaggtctaccgtataccccgtagcctcgtctgccggtccttccgcggccgcctggtctccccgtagcattctgcggccgccaggtctacccggccgcggtcgccatgtttgttttgccggttggcgacctgtaggggtcgctgcaggtccggccgcgagaacggcgttggggtctgcccggcagcgacgggagcgcgcgcgcgcgcgcgtttgcgaaggcgcgcgcccgtcggtgggtcgagcggttcccgagcggttccgatggcgcggaaagcgtcggaaattcccggaaaggtccgaaaaagcccgaaaaggtccgaaaagtgccggaagggcccgctcgaaaagcgaaggaaggcgggccggcggtgttggttcccggccggcgggtctccccgtgtcctcctgaggttgccaggtctacccgcgcgaaggagtttgccgacgatcgggcagagggaacggcggccaggtctaccgcgtctgcgacgggactttggggcccgccgtggggctggaggtagactagcagctcgtgccacggctcggaaggtgtggtaggcgtcgctgtttctctgtgcctccagttaggtcgaggtaaaagtcggttgcgctcgggtgcctcgccgcggtagcctggaaaaggacccgcgtcgtttctgaggcaaggacttttctgtccgtctat

SEQ ID NO: 5 - DNA, 2341 bp, synthetic construct
  source 1..2341: mol_type = other DNA
agcaaaagcaggtcaattatattcaatatggaaagaataaaagaactacgaaatctaatgtcgcagtctcgcacccgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcaaggacaaactttatggagtaaaatgaatgatgccggatcagaccgagtgatggtatcacctctggctgtgacatggtggaataggaatggaccaataacaaatacagttcattatccaaaaatctacaaaacttattttgaaagagtcgaaaggctaaagcatggaacctttggccctgtccattttagaaaccaagtcaaaatacgtcggagagttgacataaatcctggtcatgcagatctcagtgccaaggaggcacaggatgtaatcatggaagttgttttccctaacgaagtgggagccaggatactaacatcggaatcgcaactaacgataaccaaagagaagaaagaagaactccaggattgcaaaatttctcctttgatggttgcatacatgttggagagagaactggtccgcaaaacgagattcctcccagtggctggtggaacaagcagtgtgtacattgaagtgttgcatttgactcaaggaacatgctgggaacagatgtatactccaggaggggaagtgaggaatgatgatgttgatcaaagcttgattattgctgctaggaacatagtgagaagagctgcagtatcagcagatccactagcatctttattggagatgtgccacagcacacagattggtggaattaggatggtagacatccttaggcagaacccaacagaagagcaagccgtggatatatgcaaggctgcaatgggactgagaattagctcatccttcagttttggtggattcacatttaagagaacaagcggatcatcagtcaagagagaggaagaggtgcttacgggcaatcttcaaacattgaagataagagtgcatgagggatatgaagagttcacaatggttgggagaagagcaacagccatactcagaaaagcaaccaggagattgattcagctgatagtgagtgggagagacgaacagtcgattgccgaagcaataattgtggccatggtattttcacaagaggattgtatgataaaagcagtcagaggtgatctgaatttcgtcaatagggcgaatcaacgattgaatcctatgcatcaacttttaagacattttcagaaggatgcgaaagtgctttttcaaaattggggagttgaacctatcgacaatgtgatgggaatgattgggatattgcccgacatgactccaagcatcgagatgtcaatgagaggagtgagaatcagcaaaatgggtgtagatgagtactccagcacggagagggtagtggtgagcattgaccgttttttgagaatccgggaccaacgaggaaatgtactactgtctcccgaggaggtcagtgaaacacagggaacagagaaactgacaataacttactcatcgtcaatgatgtgggagattaatggtcctgaatcagtgttggtcaatacctatcaatggatcatcagaaactgggaaactgttaaaattcagtggtcccagaaccctacaatgctatacaataaaatggaatttgaaccatttcagtctttagtacctaaggccattagaggccaatacagtgggtttgtaagaactctgttccaacaaatgagggatgtgcttgggacatttgataccgcacagataataaaacttcttcccttcgcagccgctccaccaaagcaaagtagaatgcagttctcctcatttactgtgaatgtgaggggatcaggaatgagaatacttgtaaggggcaattctcctgtattcaactataacaaggccacgaagagactcacagttctcggaaaggatgctggcactttaactgaagacccagatgaaggcacagctggagtggagtccgctgttctgaggggattcctcattctgggcaaagaagacaagagatatgggccagcactaagcatcaatgaactgagcaaccttgcgaaaggagagaaggctaatgtgctaattgggcaaggagacgtggtgttggtaatgaaacggaaacgggactctagcatacttactgacagccagacagcgaccaaaagaattcggatggccatcaattagtgtcgaatagtttaaaaacgaccttgtttctact

SEQ ID NO: 6 - DNA, 2341 bp, synthetic construct
  source 1..2341: mol_type = other DNA
agcaaaagcaggcaaaccatttgaatggatgtcaatccgaccttacttttcttaaaagtgccagcacaaaatgctataagcacaactttcccttatactggagaccctccttacagccatgggacaggaacaggatacaccatggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaggcgatggctttccttgaggaatcccatcctggtatttttgaaaactcgtgtattgaaacgatggaggttgttcagcaaacacgagtagacaagctgacacaaggccgacagacctatgactggactctaaatagaaaccaacctgctgcaacagcattggccaacacaatagaagtgttcagatcaaatggcctcacggccaatgagtctggaaggctcatagacttccttaaggatgtaatggagtcaatgaacaaagaagaaatggggatcacaactcattttcagagaaagagacgggtgagagacaatatgactaagaaaatgataacacagagaacaatgggtaaaaagaagcagagattgaacaaaaggagttatctaattagagcattgaccctgaacacaatgaccaaagatgctgagagagggaagctaaaacggagagcaattgcaaccccagggatgcaaataagggggtttgtatactttgttgagacactggcaaggagtatatgtgagaaacttgaacaatcagggttgccagttggaggcaatgagaagaaagcaaagttggcaaatgttgtaaggaagatgatgaccaattctcaggacaccgaactttctttcaccatcactggagataacaccaaatggaacgaaaatcagaatcctcggatgtttttggccatgatcacatatatgaccagaaatcagcccgaatggttcagaaatgttctaagtattgctccaataatgttctcaaacaaaatggcgagactgggaaaagggtatatgtttgagagcaagagtatgaaacttagaactcaaatacctgcagaaatgctagcaagcatcgatttgaaatatttcaatgattcaacaagaaagaagattgaaaaaatccgaccgctcttaatagaggggactgcatcattgagccctggaatgatgatgggcatgttcaatatgttaagcactgtattaggcgtctccatcctgaatcttggacaaaagagatacaccaagactacttactggtgggatggtcttcaatcctctgacgattttgctctgattgtgaatgcacccaatcatgaagggattcaagccggagtcgacaggttttatcgaacctgtaagctacttggaatcaatatgagcaagaaaaagtcttacataaacagaacaggtacatttgaattcacaagttttttctatcgttatgggtttgttgccaatttcagcatggagcttcccagttttggggtgtctgggatcaacgagtcagcggacatgagtattggagttactgtcatcaaaaacaatatgataaacaatgatcttggtccagcaacagctcaaatggcccttcagttgttcatcaaagattacaggtacacgtaccgatgccatataggtgacacacaaatacaaacccgaagatcatttgaaataaagaaactgtgggagcaaacccgttccaaagctggactgctggtctccgacggaggcccaaatttatacaacattagaaatctccacattcctgaagtctgcctaaaatgggaattgatggatgaggattaccaggggcgtttatgcaacccactgaacccatttgtcagccataaagaaattgaatcaatgaacaatgcagtgatgatgccagcacatggtccagccaaaaacatggagtatgatgctgttgcaacaacacactcctggatccccaaaagaaatcgatccatcttgaatacaagtcaaagaggagtacttgaggatgaacaaatgtaccaaaggtgctgcaatttatttgaaaaattcttccccagcagttcatacagaagaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgtttctact

SEQ ID NO: 7 - DNA, 2233 bp, synthetic construct
  source 1..2233: mol_type = other DNA
agcaaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagattttcacttcatcaatgagcaaggcgagtcaataatcgtagaacttggtgatccaaatgcacttttgaagcacagatttgaaataatcgagggaagagatcgcacaatggcctggacagtagtaaacagtatttgcaacactacaggggctgagaaaccaaagtttctaccagatttgtatgattacaaggagaatagattcatcgaaattggagtaacaaggagagaagttcacatatactatctggaaaaggccaataaaattaaatctgagaaaacacacatccacattttctcgttcactggggaagaaatggccacaaaggcagactacactctcgatgaagaaagcagggctaggatcaaaaccagactattcaccataagacaagaaatggccagcagaggcctctgggattcctttcgtcagtccgagagaggagaagagacaattgaagaaaggtttgaaatcacaggaacaatgcgtaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaagaaaacaagtcagctaaagtgggcacttggtgagaacatggcaccagaaaaggtagactttgacgactgtaaagatgtaggtgatttgaagcaatatgatagtgatgaaccagaattgaggtcgcttgcaagttggattcagaatgagtttaacaaggcatgcgaactgacagattcaagctggatagagctcgatgagattggagaagatgtggctccaattgaacacattgcaagcatgagaaggaattatttcacatcagaggtgtctcactgcagagccacagaatacataatgaagggagtgtacatcaatactgccttgcttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacataaatgggagaagtactgtgttcttgagataggagatatgcttataagaagtgccataggccaggtttcaaggcccatgttcttgtatgtgagaacaaatggaacctcaaaaattaaaatgaaatggggaatggagatgaggcgttgcctcctccagtcacttcaacaaattgagagtatgattgaagctgagtcctctgtcaaagagaaagacatgaccaaagagttctttgagaacaaatcagaaacatggcccattggagagtcccccaaaggagtggaggaaagttccattgggaaggtctgcaggactttattagcaaagtcggtattcaacagcttgtatgcatctccacaactagaaggattttcagctgaatcaagaaaactgcttcttatcgttcaggctcttagggacaacctggaacctgggacctttgatcttggggggctatatgaagcaattgaggagtgcctgattaatgatccctgggttttgcttaatgcttcttggttcaactccttccttacacatgcattgagttagttgtggcagtgctactatttgctatccatactgtccaaaaaagtaccttgtttctact

SEQ ID NO: 8 - DNA, 1565 bp, synthetic construct
  source 1..1565: mol_type = other DNA
agcaaaagcagggtagataatcactcactgagtgacatcaaaatcatggcgtctcaaggcaccaaacgatcttacgaacagatggagactgatggagaacgccagaatgccactgaaatcagagcatccgtcggaaaaatgattggtggaattggacgattctacatccaaatgtgcaccgaactcaaactcagtgattatgagggacggttgatccaaaacagcttaacaatagagagaatggtgctctctgcttttgacgaaaggagaaataaataccttgaagaacatcccagtgcggggaaagatcctaagaaaactggaggacctatatacaggagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagagctcttgttcgcaccggaatggatcccaggatgtgctctctgatgcaaggttcaactctccctaggaggtctggagccgcaggtgctgcagtcaaaggagttggaacaatggtgatggaattggtcagaatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccggaacccagggaatgctgagttcgaagatctcacttttctagcacggtctgcactcatattgagagggtcggttgctcacaagtcctgcctgcctgcctgtgtgtatggacctgccgtagccagtgggtacgactttgaaagggagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattaagcttcatcaaagggacgaaggtgctcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccattatggcagcattcaatgggaatacagaggggagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

(19) United States
(12) Patent Application Publication
(10) Pub. No.: US 2025/0320466 A1
(43) Pub. Date: Oct. 16, 2025

(54) DUCK-DERIVED RNA POLYMERASE I PROMOTER AND RECOMBINANT VECTOR CARRYING SAME

(71) Applicant: REPUBLIC OF KOREA(ANIMAL AND PLANT QUARANTINE AGENCY), Gyeongsangbuk-do (KR)

(72) Inventors: Yu-Na LEE, Gyeongsangbuk-do (KR); Eun-Kyoung LEE, Gyeongsangbuk-do (KR); Ra Mi CHA, Gyeongsangbuk-do (KR); Yeon Chan CHOI, Gyeongsangbuk-do (KR); Youn-Jeong LEE, Gyeongsangbuk-do (KR)

(21) Appl. No.: 18/866,156

(22) PCT Filed: Feb. 14, 2024
(86) PCT No.: PCT/KR2024/002088

§ 371 Date: Nov. 15, 2024

(30) Foreign Application Priority Data
Mar. 8, 2023 (KR) 10-2023-0030832

(51) Int. Cl. C12N 7/00; C12N 9/12

(52) U.S. Cl. CPC C12N 7/00; C12N 9/1247; C12Y 207/07006; C12N 2760/16152

(57) ABSTRACT

The present disclosure relates to a duck-derived RNA polymerase I promoter and a recombinant vector comprising the same. The use of the duck-derived RNA polymerase I promoter of the present disclosure enables the production of avian influenza viruses with high efficiency. Furthermore, In the event of the emergence of the variant and novel avian influenza viruses, vaccine candidate libraries and diagnostic standards can be early ensured on the basis of the virus production system, thereby minimizing damage to poultry industry.

### TECHNICAL FIELD

The present disclosure was made with the support of the Ministry of Agriculture, Food, and Rural Affairs, Republic of Korea, under Project No. B-1543418-2022-23-01, which was conducted in the research project named “Development of Avian RNA Polymerase I-Driven Reverse Genetics System for The Rescue of Avian Influenza Virus” in the research program titled “Development of Animal and Plant Quarantine and Inspection Technology” by the Animal and Plant Quarantine Agency, under management of the Animal and Plant Quarantine Agency, from 1 Jan. 2022 to 31 Dec. 2023.

This application claims priority and the benefit of Korean Patent Application No. 10-2023-0030832 filed in the Korean Intellectual Property Office on 8 Mar. 2023, the disclosure of which is incorporated herein by reference.

The present disclosure relates to a duck-derived RNA polymerase I promoter and a recombinant vector comprising the same.

### BACKGROUND ART

H5 subtype high pathogenicity avian influenza (HPAI) viruses first attracted global attention in 1997 when the viruses were first directly transmitted from poultry to humans in Hong Kong. A/Goose/Guangdong/1/1996 (Gs/GD) lineage of HPAI in 1997, H5 subtype viruses have become enzootic in some countries, have circulated in wild birds and poultry, and caused sporadic human infections. HPAI H5 viruses have been evolving into genetically diverse clades and subclades. Since the early 2000s, spillover of H5 subtype viruses has occurred in subsequent inter and trans-continental spread of H5 subtype viruses via wild bird migrations.

Since 2003, South Korea has also experienced enormous social and economic damage due to outbreaks of H5N1, H5N8, and H5N6 HPAI, and is likely to face a reintroduction of the disease in the future. In particular, the outbreak of the

H5N6 HPAI viruses during 2016/2017 caused the unprecedented outbreaks of HPAI in poultry farm in a short period of time, resulting in the establishment of an avian influenza national antigen bank for emergency preparedness in 2018. Currently, H5 avian influenza vaccine strains for the antigen bank are produced using reverse genetics systems. The influenza recombinant virus production technology that has been currently used worldwide is based on reverse genetics systems using human-derived promoters (Hoffmann et al., 2000).

Since research on human-derived influenza viruses and vaccine production is actively conducted, optimized reverse genetics systems for human-derived influenza virus production have been established. However, the reverse genetics systems optimized for the Human influenza virus have limitations in producing recombinant viruses for avian influenza virus vaccine production and identifying genetic markers related to pathogenicity and the like. There is thus a need to develop vectors comprising avian-derived promoters that operate in avian cells.

### DISCLOSURE OF INVENTION

### Technical Problem

The present inventors have made intensive efforts to develop an efficient recombinant virus production system for the production of vaccines against avian influenza viruses. As a result, the present inventors established that the use of a polymerase I promoter derived from ducks can effectively produce recombinant avian influenza viruses compared with the use of a promoter derived from humans, and thus completed the present disclosure.

Accordingly, an aspect of the present disclosure is to provide a nucleic acid molecule comprising a nucleotide sequence selected from the group consisting of SEQ ID NOS: 1 to 4.

Another aspect of the present disclosure is to provide a recombinant vector comprising the nucleic acid molecule.

Still another aspect of the present disclosure is to provide a host cell comprising the recombinant vector.

Still another aspect of the present disclosure is to provide a method for producing a virus by culturing a host cell transformed with a recombinant vector comprising the nucleic acid molecule.

Other purposes and advantages of the present disclosure will become more obvious when taken with the following detailed description of the invention, claims, and drawings.

### Solution to Problem

In accordance with an aspect of the present disclosure, there is provided a nucleic acid molecule comprising the nucleotide sequence represented by SEQ ID NO: 1.

The present inventors have made intensive efforts to develop an efficient recombinant virus production system for the production of vaccines against avian influenza viruses. As a result, the present inventors established that the use of a polymerase I promoter derived from ducks can effectively produce recombinant avian influenza viruses compared with the use of a promoter derived from humans.

As used herein, the term “nucleic acid molecule” comprehensively encompasses DNA (gDNA and cDNA) and RNA molecules, and the nucleotide, which is a basic constituent of the nucleic acid molecule, include not only natural nucleotides but also analogues having modified sugar or base moieties (Scheit, Nucleotide Analogs, John Wiley, New York (1980); and Uhlman and Peyman, Chemical Reviews, 90:543-584 (1990)).

The nucleic acid molecule including a nucleotide sequence is also construed to include nucleotide sequences showing substantial identity to the nucleotide sequence. The substantial identity denotes at least 61% homology, more preferably 70% homology, still more preferably 80% homology, most preferably 90% homology in sequences when the present nucleotide sequence is aligned with any other sequence so as to match each other as much as possible and the aligned sequences are analyzed using an algorithm commonly used in the art. Methods of the alignment for sequence comparison are known in the art. Various methods and algorithms for alignment are disclosed in Smith and Waterman, Adv. Appl. Math. 2: 482 (1981); Needleman and Wunsch, J. Mol. Bio. 48: 443 (1970); Pearson and Lipman, Methods in Mol. Biol. 24:307-31 (1988); Higgins and Sharp, Gene 73:237-44 (1988); Higgins and Sharp, CABIOS 5:151-3 (1989); Corpet et al., Nuc. Acids Res. 16:10881-90 (1988); Huang et al., Comp. Appl. BioSci. 8:155-65 (1992); and Pearson et al., Meth. Mol. Biol. 24:307-31 (1994). The NCBI Basic Local Alignment Search Tool (BLAST) (Altschul et al., J. Mol. Biol. 215:403-10 (1990)) is accessible from the National Center for Biotechnology Information (NCBI) and, on the Internet, may be used in connection with sequence analysis programs, such as blastp, blastn, blastx, tblastn and tblastx. BLAST is accessible through the BLAST page of the NCBI website. Sequence homology comparison methods using this program can be found on the BLAST help page of the NCBI website.

In an embodiment of the present disclosure, the nucleic acid molecule comprises a nucleotide sequence represented by any one of SEQ ID NOS: 2 to 4.

In an embodiment of the present disclosure, the nucleic acid molecule is an RNA polymerase I promoter derived from duck (Anas platyrhynchos). Anas platyrhynchos, called the mallard duck, is one of the most commonly observed ducks in South Korea. Anas platyrhynchos is a migratory bird and is also a cause of transmitting the avian influenza virus.

RNA polymerase (RNAP or RNApol) is also called an RNA polymerizing enzyme and is an enzyme that synthesizes primary transcript RNA from DNA. RNA polymerase is essential for the transcription process that forms a RNA chain by using DNA, and thus is found in all living organisms and many viruses. There are several types of eukaryotic nuclear RNA polymerases, which are used to synthesize different types of RNAs, respectively, and are similar to bacterial RNA polymerases in terms of structure and mechanism. RNA polymerase I synthesizes the precursor (pre-rRNA) 45S. The precursor rRNA matures into 28S, 18S and 5.8S rRNAs, which are major RNAs constituting the ribosome.

As used herein, the term “promoter” refers to the upstream region of a gene that is involved in the start of transcription. That is, the promoter indicates a site of the DNA strand to which RNA polymerase binds, which is typically located within 1 kbps from the start of a specific gene.

In accordance with an aspect of the present disclosure, there is provided a recombinant vector comprising the nucleic acid molecule.

As used herein, the term “vector” refers to a means for expressing a target gene in a host cell, and encompasses: plasmid vectors; cosmid vectors; and viral vectors, such as bacteriophage vectors, adenoviral vectors, retroviral vectors, and adeno-associated viral vectors.

The recombinant vector system of the present disclosure may be constructed by various methods known in the art, and a specific method therefor is disclosed in Sambrook et al., Molecular Cloning, A Laboratory Manual, Cold Spring Harbor Laboratory Press (2001), which is incorporated herein by reference.

The vector of the present disclosure may be typically constructed as a vector for gene cloning or a vector for protein expression. In addition, the vector of the present disclosure may be constructed by using a prokaryotic or eukaryotic cell as a host.

As used herein, the term gene refers to a transcription unit and regulatory regions that are flanked by (e.g., located upstream and downstream) and operably linked to the transcription unit. The transcription unit is a series of nucleotides that are transcribed into an RNA molecule. The transcription unit may comprise a coding region. The “coding region” is a nucleotide sequence that encodes an unprocessed preRNA (i.e., an RNA molecule that comprises both exons and introns) that is subsequently processed to an mRNA. The transcription unit may encode a non-coding RNA. The non-coding RNA is an RNA molecule that is not translated into a protein. Examples of non-coding RNAs include microRNA. The boundaries of the transcription unit are generally determined by an initiation site at its 5′ end and a transcription terminator at its 3′ end. A “regulatory region” is a nucleotide sequence that regulates expression of a transcription unit to which it is operably linked. Non-limiting examples of regulatory sequences include promoters, enhancers, transcription initiation sites, translation start sites, translation stop sites, transcription terminators, and poly(A) signals. A regulatory region located upstream of the transcription unit may be referred to as a 5′ UTR, and a regulatory region located downstream of the transcription unit may be referred to as a 3′ UTR. A regulatory region may be transcribed and be part of an unprocessed preRNA.

The present disclosure also provides a vector comprising the promoter of the present disclosure that is operably linked to a coding sequence. The coding sequence may be, for example, a sequence encoding the whole gene or a predetermined region thereof. As used herein, the term “operably linked” indicates that the coding sequence is functionally linked to the promoter such that the promoter sequence can initiate or mediate transcription of the coding sequence. The vector of the present disclosure may further comprise 5′ and 3′ control sequences operably linked to the promoter sequence.

The vector of the present disclosure may be fused with the other sequences to facilitate the purification of polypeptides or proteins expressed therefrom. Examples of the fused sequence include glutathione S-transferase (Pharmacia, USA), maltose-binding proteins (NEB, USA), FLAG (IBI, USA), 6-His (hexahistidine; Quiagen, USA), and the like.

Meanwhile, the expression vector of the present disclosure comprises, as a selective marker, an antibiotic agent-resistant gene that is commonly used in the art, and examples thereof include resistant genes against ampicillin, gentamycin, carbenicillin, chloramphenicol, streptomycin, kanamycin, geneticin, neomycin, and tetracycline.

In an embodiment of the present disclosure, the vector further comprises a gene selected from the group consisting of PB2, PB1, PA, HA, NP, NA, M, NS, and a combination thereof.

Influenza viruses are negative strand RNA viruses belonging to the Orthomyxoviridae family. The influenza A and B virus genomes comprise eight single-stranded viral RNA (vRNA) segments. The eight genomic segments of the influenza A and B viruses are, in order of size, PB2, PB1, PA, HA, NP, NA, M, and NS. The PB2, PB1, PA, NP, M and NS encode the internal and non-structural proteins, and may be referred to as backbone segments. The HA and NA segments encode surface glycoproteins. The method of the present disclosure may be used to produce reassortant influenza A viruses. Alternatively, the methods of the invention may be used to produce reassortant influenza B viruses.

For influenza A viruses, the eight genome segments encode eleven proteins, as follows: haemagglutinin (HA), neuraminidase (NA), two matrix proteins (M1 and M2), a heterotrimeric RNA-dependent RNA polymerase (made up of one polymerase acidic subunit (PA), and two polymerase basic subunits (PB1 and PB2)), nucleoprotein (NP), and two non-structural proteins (NS1 and NS2; NS2 is also known as nuclear export protein (NEP)). Some influenza A viruses also express a pro-apoptotic peptide, PB1-F2. The PB2, PA, HA, NP and NA segments each encode a single expressed protein. The PB1, M, and NS segments encode more than one protein. The PB1 segment encodes the PB1 protein, as well as the PB1-F2 protein (which is encoded in a +1 reading frame on the PB1 segment). The M segment encodes the M1 and M2 proteins. The NS segment encodes the NS1 and NS2/NEP proteins. The M2 and NS2/NEP protein are expressed from spliced mRNAs from the M and NS segments.

For influenza B viruses, the eight genome segments also encode eleven proteins, but there are a few differences from influenza A viruses. As for influenza A viruses, the PB2, PA, HA and NP segments each encode a single expressed protein, and the NS segment encodes the NS1 and NS2/NEP protein. The PB1-F2 protein is not present in influenza B viruses, meaning that the PB1 segment encodes the PB1 protein alone. In addition to the NA protein, the NA segment in influenza B viruses also encodes the NB matrix protein in an alternate-1 reading frame. The NB matrix protein corresponds to the M2 protein in influenza A. The M segment encodes the M1 protein, and in an alternate +2 reading frame, the BM2 protein.

In an embodiment of the present disclosure, the viral vector may be (a) a vector selected from: a vector comprising a nucleotide sequence wherein a promoter, an influenza virus PA cDNA, and a transcription termination sequence are operably linked; a vector comprising a nucleotide sequence wherein a promoter, an influenza virus PB1 cDNA, and a transcription termination sequence are operably linked; a vector comprising a nucleotide sequence wherein a promoter, an influenza virus PB2 cDNA, and a transcription termination sequence are operably linked; a vector comprising a nucleotide sequence wherein a promoter, an influenza virus HA cDNA, and a transcription termination sequence are operably linked; a vector comprising a nucleotide sequence wherein a promoter, an influenza virus NP cDNA, and a transcription termination sequence are operably linked; a vector comprising a nucleotide sequence wherein a promoter, an influenza virus NA cDNA, and a transcription termination sequence are operably linked; a vector comprising a nucleotide sequence wherein a promoter, an influenza virus M cDNA, and a transcription termination sequence are operably linked; and a vector comprising a nucleotide sequence wherein a promoter, an influenza virus NS cDNA, and a transcription termination sequence are operably linked, and b) a vector selected from a vector encoding influenza virus PA, a vector selected from a vector encoding influenza virus PB1, a vector selected from a vector encoding influenza virus PB2, and a vector selected from a vector encoding influenza virus NP.

As such, the present disclosure provides an isolated and purified vector or plasmid expressing or encoding an influenza virus protein or expressing or encoding an influenza vRNA. Therefore, the vector or plasmid of the present disclosure may comprise an exogenous gene encoding a gene of interest or an open reading frame of interest, for example an immunogenic peptide or protein useful as a vaccine. For vectors or plasmids comprising a gene or open reading frame of interest, it is preferred that the gene or open reading frame is flanked by 5′ and 3′ influenza virus sequences (comprising 5′ and 3′ non-coding sequences, respectively) of influenza virus, and in one embodiment, that the gene or open reading frame flanked by 5′ and 3′ influenza virus sequences is operably linked to a RNA polymerase I promoter and a RNA polymerase I transcription termination sequence. When preparing a virus, the vector or plasmid comprising the gene or open reading frame may substitute for a vector or plasmid for an influenza viral gene or may be in addition to vectors or plasmids for all influenza viral genes.

A plurality of the vectors of the present disclosure may be physically linked or each vector may be present on an individual plasmid or other (e.g., linear) nucleic acid delivery vehicle.

In accordance with an aspect of the present disclosure, there is provided a host cell comprising the recombinant vector.

In an embodiment of the present disclosure, the host cell is transformed with the recombinant vector.

As used herein, the term “transformed”, “transduced”, or “transinfected” refers to a process for delivering or introducing an exogenous nucleic acid into a host cell. A “transformed”, “transduced”, or “transfected” cell is a cell that has been transformed, transduced, or transfected with an exogenous nucleic acid, and the cell includes the primary subject cell and its progenies resulting from passages.

In an embodiment of the present disclosure, the host cell is an avian cell.

As used herein, the terms “avian”, “bird”, “aves”, or “ava” is intended to have the same meaning, and will be used indistinctly. The “bird” refer to any species, subspecies, or breed of organism of the taxonomic class “ava”. In a preferred embodiment, the “bird” refers to any animal of the taxonomic order.

The bird may be, for example, a chicken, a duck, or a quail, but is not limited thereto, and includes any bird capable of providing a host cell. Preferably, the bird may be a duck. Examples of the host cell may include avian cells, such as chicken embryo fibroblasts (CEFs), QT-35 cells, QM-7 cells or chicken hepatoma cell line (LMH), DF-1 cells, CCL-141 cells, and QT-6 cells. However, the host cell is not limited thereto, and any host cell that can stably and continuously express the vector of the present disclosure may be used. Examples of the host cells may include monkey kidney cell 7 (COS7), NSO cells, SP2/0, Chinese hamster ovary (CHO) cells, W138, baby hamster kidney (BHK) cells, MDCK, myeloma cell lines, HuT 78 cells, and HEK-293 cells, but are not limited thereto.

In accordance with an aspect of the present disclosure, there is provided a method for producing a virus by culturing a host cell comprising the nucleic acid molecule or a recombinant vector.

The term “producing a virus” may refer to the production of live viruses, attenuated viruses, and/or virus-like particles (VLPs). The production may be performed by a general method, including the production in 1) an organism (e.g., egg yolk), cultured cells (e.g., avian cells), or in vitro (e.g., cell lysate).

In an embodiment of the present disclosure, examples of the virus that can be produced using the present disclosure may include avian influenza virus (AIV), infectious bronchitis virus (IBV), infectious bursal disease virus (IBDV), Marek's disease virus (MDV), avian metapneumovirus (aMPV), infectious bursal disease virus (IBDV), or Newcastle disease virus (NDV). However, the virus is not limited thereto, and encompasses any virus that can be produced using avian cells as host cells. Preferably, the virus may be an avian influenza virus.

In an embodiment of the present disclosure, the present disclosure comprises the steps of: providing a host cell transformed with a recombinant vector comprising the avian-derived promoter; incubating the host cell under conditions suitable for producing viruses by cells; and optionally collecting the viruses produced by the cells.

In accordance with another aspect of the present disclosure, there is provided a composition further comprising a vector comprising a promoter linked to 5′ viral sequences, which May 5′ viral coding sequences or a portion thereof, wherein the sequence is linked to a desired nucleic acid sequence (e.g., a desired cDNA) linked to 3′ viral sequence linked to a transcription termination sequence. Preferably, the desired nucleic acid sequence, such as cDNA, is in an antisense orientation. The introduction of the composition into a host cell permissive for viral replication results in a recombinant virus comprising vRNA corresponding to sequences of the vector comprising 5′ viral sequences linked to the cDNA linked to 3′ viral sequences.

### Advantageous Effects of Invention

Features and advantages of the present disclosure are summarized as follows.
(a) The present disclosure provides a nucleic acid molecule comprising a nucleotide sequence selected from the group consisting of SEQ ID NOS: 1 to 4.
(b) The present disclosure provides a recombinant vector c comprising the nucleic acid molecule.
(c) The present disclosure provides a host cell comprising the recombinant vector.
(d) The present disclosure provides a method for producing a virus by culturing a host cell comprising the nucleic acid molecule or a recombinant vector.
(e) The use of the duck-derived RNA polymerase I promoter of the present disclosure enables the production of avian influenza viruses with high efficiency. Furthermore, in the event of outbreaks of mutant avian influenza viruses and new avian influenza viruses, vaccine candidate libraries and diagnostic solutions can be early ensured on the basis of the virus production system, thereby minimizing damage to poultry industry.

### BRIEF DESCRIPTION OF DRAWINGS

FIG. 1 is a schematic diagram comparing the nucleotide sequences from −8 to +11 with reference to the transcription start site (+1) of RNA polymerase I among different species.

FIG. 2 is a schematic diagram of reporter plasmids constructed by inserting duck-derived promoters with 250, 500, 750, and 1,000 nucleotides in length into the pUC57 vectors comprising the luciferase gene and the polymerase I terminator gene, respectively.

FIG. 3A is a vector map of reporter plasmids constructed by inserting duck-derived promoters with 250, 500, 750, and 1,000 nucleotides in length into the pUC57 vector comprising the luciferase gene and the polymerase I terminator gene, respectively.

FIG. 3B is a schematic diagram illustrating a procedure for measuring the activity of luciferase by introducing the constructed reporter plasmid vectors into DF-1 and QT6 cells.

FIG. 4 shows the results of comparing the activity of luciferase according to the length of the duck-derived promoter by measuring the expression level of luciferase in DF-1 and QT6 cells.

### MODE FOR CARRYING OUT THE INVENTION

Hereinafter, the present disclosure will be described in more detail with reference to exemplary embodiments. These exemplary embodiments are provided only for the purpose of specifically illustrating the present disclosure, and therefore, according to the purpose of the present disclosure, it would be apparent to a person skilled in the art that these exemplary embodiments are not construed to limit the scope of the present disclosure.

### EXAMPLES

### Example 1: Search of Duck-Derived RNA Polymerase I Promoter

The sequence (GenBank accession no. NW_024010378.1) corresponding to scaffold 92 among the entire shotgun sequences of duck (Anas platyrhynchos) published in the NCBI Genbank database was compared and analyzed with the 18S rRNA sequence (GenBank accession no. XR_003493879) and 28S rRNA sequence (GenBank accession no. XR_003493880) of duck (Anas platyrhynchos) to predict a duck RNA polymerase I transcription start region. Based on the prediction, a duck RNA polymerase I promoter region was analyzed.

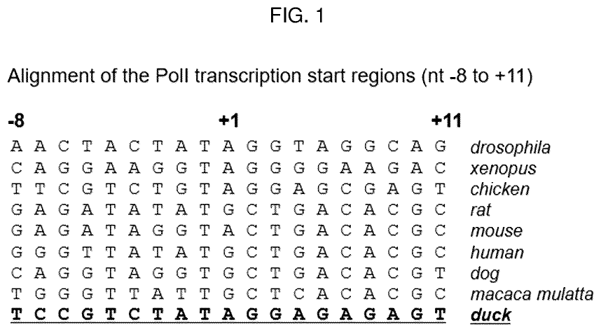

The results are shown FIG. 1.

As shown in FIG. 1, a similar sequence regularity to the RNA polymerase I promoter of chicken, which is derived from the same avian group, was identified around the transcription start region.

### Example 2: Obtaining Duck-Derived RNA Polymerase I Promoter Genes and Constructing Plasmids

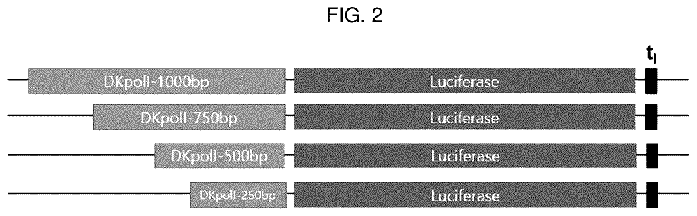

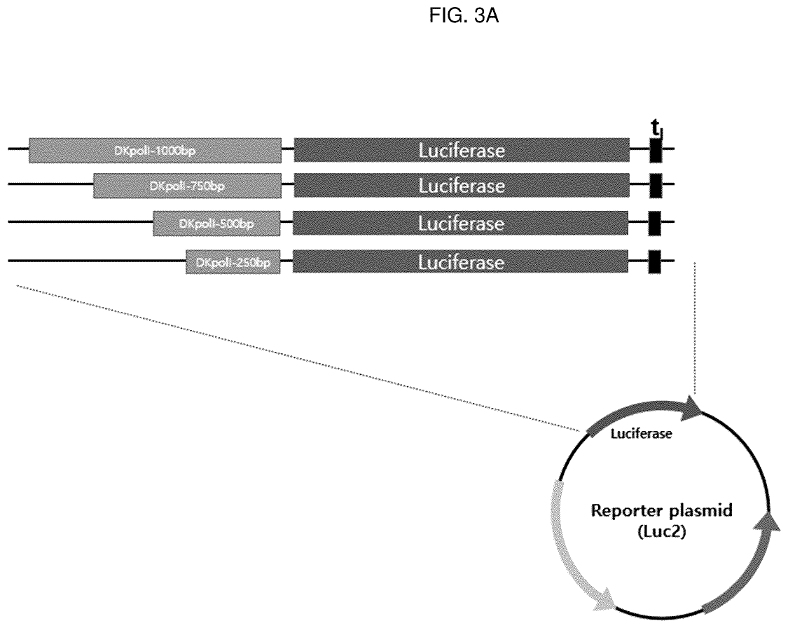

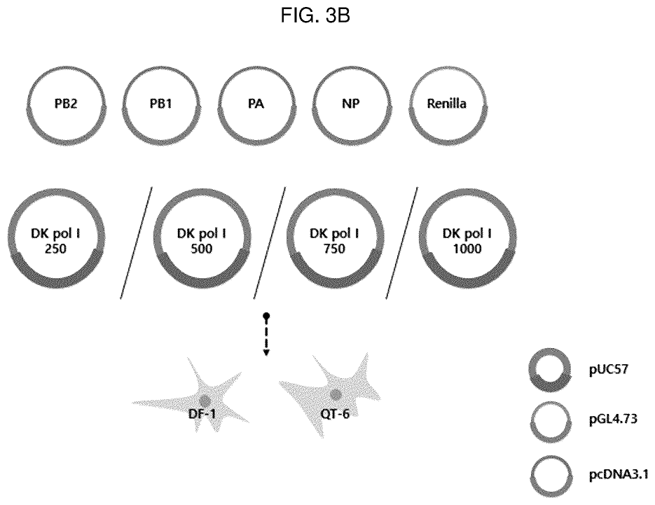

To compare the change in activity according to the promoter length, four types of promoters with nucleotide lengths of 250, 500, 750, and 1,000 bp were selected (SEQ ID NOS: 1 to 4). Each promoter was obtained through gene synthesis. Gene synthesis was carried out by Genscript (Piscataway, New Jersey, USA). As shown in FIGS. 2 and 3A, the promoter genes obtained through the synthesis were each inserted into the pUC57 vector comprising the luciferase gene and the polymerase I terminator gene, thereby completing reporter plasmids.

### Example 3: Evaluation of Activity of Duck-Derived RNA Polymerase I Promoters

The promoter activity was evaluated by measuring the luciferase expression level in the four types of plasmids produced in Example 2. The procedure for luciferase expression measurement is shown in FIG. 3B. In addition to the four types of reporter plasmids, plasmids in which PB2, PB1, PA, and NP genes of the A/PR/8/34 virus were inserted into the pcDNA3.1 (+) vector were used to utilize the ribonucleoproteins (RNPs) of the influenza virus. Additionally, for relative quantification, pGL4.73 (Promega, USA) expressing Renilla luciferase was used. Specifically, to examine the promoter activity of the reporter plasmids, RNP expression plasmid, pGL4.73, and four types of reporter plasmids were transfected in DF-1, a chicken embryonic fibroblast cell line, and QT-6, a quail fibroblast cell line, and then the expression level of luciferase was measured after 48 hours. The expression level of luciferase was repeatedly measured nine times.

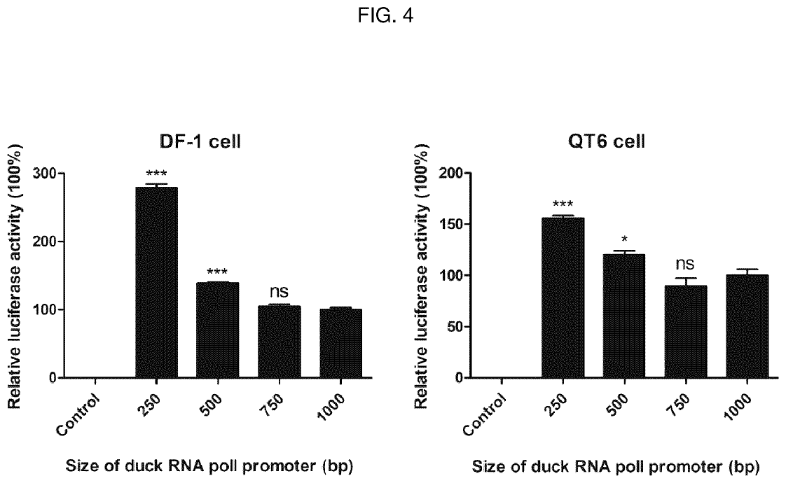

The results are shown in FIG. 4 and Tables 1 and 2.

As shown in FIG. 4 and Table 1, a total of four types of duck-derived RNA polymerase I promoters with 250 bp to 1,000 bp in length were capable of luciferase expression in DF-1 and QT-6 cells. Especially, the duck-derived polymerase I promoter with a length of 250 bp showed higher expression efficiency compared with the duck-derived polymerase I promoters with the other lengths. Specifically, the duck-derived polymerase I promoter with a length of 250 bp showed a luciferase expression level about 30% to 300% higher than those of the duck-derived polymerase I promoters with lengths of 500 to 1000 bp.

TABLE 1
Relative comparison of luciferase expression levels
for different promoter lengths in DF-1 cell line

| | Control | 250-bp | 500-bp | 750-bp | 1000-bp |
|---|---|---|---|---|---|
| | group | promoter | promoter | promoter | promoter |
| Mean | 0.06 | 278.95 | 138.81 | 104.66 | 100.00 |
| Standard | 0.09 | 15.88 | 4.56 | 10.64 | 8.91 |
| deviation | | | | | |

TABLE 2
Relative comparison of luciferase expression levels
for different promoter lengths in QT-6 cell line

| | Control | 250-bp | 500-bp | 750-bp | 1000-bp |
|---|---|---|---|---|---|
| | group | promoter | promoter | promoter | promoter |
| Mean | 0.07 | 156.11 | 120.25 | 89.81 | 100.00 |
| Standard | 0.01 | 6.91 | 11.47 | 22.36 | 17.71 |
| deviation | | | | | |

1. A nucleic acid molecule comprising the nucleotide sequence represented by SEQ ID NO: 1.

2. The nucleic acid molecule of claim 1, wherein the nucleic acid molecule comprises a nucleotide sequence represented by any one of SEQ ID NOS: 2 to 4.

3. The nucleic acid molecule of claim 1, wherein the nucleic acid molecule is an RNA polymerase I promoter derived from duck (Anas platyrhynchos).

4. A recombinant vector comprising the nucleic acid molecule of claim 1.

5. The recombinant vector of claim 4, further comprising a gene selected from the group consisting of PB2, PB1, PA, HA, NP, NA, M, NS, and a combination thereof.

6. A host cell comprising the recombinant vector of claim 4.

7. The host cell of claim 6, wherein the host cell is an avian cell.

8. A method for producing a virus by culturing a host cell comprising the nucleic acid molecule of claim 1.

9. A method for producing a virus, comprising:
culturing a host cell comprising the recombinant vector of claim 4.
